SEQUENCE LISTING (5 sequences)

SEQ ID NO: 1 - protein, 589 aa, Artificial Sequence
  Poly(3-hydroxyalkanoate) polymerase (PHA synthase)
MATGKGAAASTQEGKSQPFKVTPGPFDPATWLEWSRQWQGTEGNGHAAASGIPGLDALAGVKIAPAQLGDIQQRYMKDFSALWQAMAEGKAEATGPLHDRRFAGDAWRTNLPYRFAAAFYLLNARALTELADAVEADAKTRQRIRFAISQWVDAMSPANFLATNPEAQRLLIESGGESLRAGVRNMMEDLTRGKISQTDESAFEVGRNVAVTEGAVVFENEYFQLLQYKPLTDKVHARPLLMVPPCINKYYILDLQPESSLVRHVVEQGHTVFLVSWRNPDASMAGSTWDDYIEHAAIRAIEVARDISGQDKINVLGFCVGGTIVSTALAVLAARGEHPAASVTLLTTLLDFADTGILDVFVDEGHVQLREATLGGGAGAPCALLRGLELANTFSFLRPNDLVWNYVVDNYLKGNTPVPFDLLFWNGDATNLPGPWYCWYLRHTYLQNELKVPGKLTVCGVPVDLASIDVPTYIYGSREDHIVPWTAAYASTALLANKLRFVLGASGHIAGVINPPAKNKRSHWTNDALPESPQQWLAGAIEHHGSWWPDWTAWLAGQAGAKRAAPANYGNARYRAIEPAPGRYVKAKA

SEQ ID NO: 2 - protein, 156 aa, Artificial Sequence
  (R)-specific enoyl-CoA hydratase
MSQVTNTPYEALEVGQKAEYKKSVEERDIQLFAAMSGDHNPVHLDAEFAAKSMFRERIAHGMFSGALISAAVACTLPGPGTIYLGQQMSFQKPVKIGDTLTVRLEILEKLPKFKVRIATNVYNQNDELVVAGEAEILAPRKQQTVELVSPPNFVAS

SEQ ID NO: 3 - protein, 814 aa, Artificial Sequence [sequence omitted]

SEQ ID NO: 4 - DNA, 470 bp, Artificial Sequence
  Coding sequence of PhaJ gene from P. putida KT2440
atgagccaggtgaccaacaccccgtacgaggccctggaggtgggccagaaggccgagtacaagaagagcgtggaggagcgcgacatccagctgttcgccgccatgagcggcgaccacaacccggtgcacctggacgccgagttcgccgccaagagcatgttcgcgagcgcatcgcccacggcatgttcagcggcgccctgatcagcgccgccgtggcctgcaccctgccgggcccgggcaccatctacctgggccagcagatgagcttccagaagccggtgaagatcggcgacaccctgaccgtgcgcctggagatcctggagaagctgccgaagttcaaggtgcgcatcgccaccaacgtgtacaaccagaacgacgagctggtggtggccggcgaggccgagatcctggccccgcgcaagcagcagaccgtggagctggtgagcccgccgaacttcgtggccagctga

SEQ ID NO: 5 - DNA, 2445 bp, Artificial Sequence
  Coding sequence of FadE gene from E. coli K12
atgatgatcctgagcatcctggccaccgtggtgctgctgggcgccctgttctaccaccgcgtgagcctgttcatcagcagcctgatcctgctggcctggaccgccgccctgggcgtggccggcctgtggagcgcctgggtgctggtgccgctggccatcatcctggtgccgttcaacttcgccccgatgcgcaagagcatgatcagcgccccggtgttccgcggcttccgcaaggtgatgccgccgatgagccgcaccgagaaggaggccatcgacgccggcaccacctggtgggagggcgacctgttccagggcaagccggactggaagaagctgcacaactacccgcagccgcgcctgaccgccgaggagcaggccttcctggacggcccggtggaggaggcctgccgcatggccaacgacttccagatcacccacgagctggccgacctgccgccggagctgtgggcctacctgaaggagcaccgcttcttcgccatgatcatcaagaaggagtacggcggcctggagttcagcgcctacgcccagagccgcgtgctgcagaagctgagcggcgtgagcggcatcctggccatcaccgtgggcgtgccgaacagcctgggcccgggcgagctgctgcagcactacggcaccgacgagcagaaggaccactacctgccgcgcctggcccgcggccaggagatcccgtgcttcgccctgaccagcccggaggccggcagcgacgccggcgccatcccggacaccggcatcgtgtgcatgggcgagtggcagggccagcaggtgctgggcatgcgcctgacctggaacaagcgctacatcaccctggccccgatcgccaccgtgctgggcctggccttcaagctgagcgacccggagaagctgctgggcggcgccgaggacctgggcatcacctgcgccctgatcccgaccaccaccccgggcgtggagatcggccgccgccacttcccgctgaacgtgccgttccagaacggcccgacccgcggcaaggacgtgttcgtgccgatcgactacatcatcggcggcccgaagatggccggccagggctggcgcatgctggtggagtgcctgagcgtgggccgcggcatcaccctgccgagcaacagcaccggcggcgtgaagagcgtggccctggccaccggcgcctacgcccacatccgccgccagttcaagatcagcatcggcaagatggagggcatcgaggagccgctggcccgcatcgccggcaacgcctacgtgatggacgccgccgccagcctgatcacctacggcatcatgctgggcgagaagccggccgtgctgagcgccatcgtgaagtaccactgcacccaccgcggccagcagagcatcatcgacgccatggacatcaccggcggcaagggcatcatgctgggccagagcaacttcctggcccgcgcctaccagggcgccccgatcgccatcaccgtggagggcgccaacatcctgacccgcagcatgatgatcttcggccagggcgccatccgctgccacccgtacgtgctggaggagatggaggccgccaagaacaacgacgtgaacgccttcgacaagctgctgttcaagcacatcggccacgtgggcagcaacaaggtgcgcagcttctggctgggcctgacccgcggcctgaccagcagcaccccgaccggcgacgccaccaagcgctactaccagcacctgaaccgcctgagcgccaacctggccctgctgagcgacgtgagcatggccgtgctgggcggcagcctgaagcgccgcgagcgcatcagcgcccgcctgggcgacatcctgagccagctgtacctggccagcgccgtgctgaagcgctacgacgacgagggccgcaacgaggccgacctgccgctggtgcactggggcgtgcaggacgccctgtaccaggccgagcaggccatggacgacctgctgcagaacttcccgaaccgcgtggtggccggcctgctgaacgtggtgatcttcccgaccggccgccactacctggccccgagcgacaagctggaccacaaggtggccaagatcctgcaggtgccgaacgccacccgcagccgcatcggccgcggccagtacctgaccccgagcgagcacaacccggtgggcctgctggaggaggccctggtggacgtgatcgccgccgacccgatccaccagcgcatctgcaaggagctgggcaagaacctgccgttcacccgcctggacgagctggcccacaacgccctggtgaagggcctgatcgacaaggacgaggccgccatcctggtgaaggccgaggagagccgcctgcgcagcatcaacgtggacgacttcgacccggaggagctggccaccaagccggtgaagctgccggagaaggtgcgcaaggtggaggccgcctaa

(19) United States
(12) Patent Application Publication
(10) Pub. No.: US 2024/0060099 A1
(43) Pub. Date: Feb. 22, 2024

(54) POLY-3-HYDROXYALKANOATES HAVING VINYL MOIETIES AND METHOD OF PRODUCING SUCH

(71) Applicant: POLYFERM CANADA INC., Harrowsmith (CA)

(72) Inventors: Bruce RAMSAY, Harrowsmith (CA); Juliana RAMSAY, Harrowsmith (CA); Araceli FLORES-SANCHEZ, Harrowsmith (CA)

(21) Appl. No.: 18/030,970

(22) PCT Filed: Oct. 11, 2021
(86) PCT No.: PCT/IB2021/000690

§ 371 Date: Apr. 7, 2023

(30) Foreign Application Priority Data
Oct. 9, 2020 (EP) 20201070.8

(51) Int. Cl. C12P 7/625; C12N 9/02; C12N 9/10; C12N 9/88; C12N 15/74

(52) U.S. Cl. CPC C12P 7/625; C12N 9/001; C12N 9/1029; C12N 9/88; C12N 15/74; C12N 2500/30; C12Y 103/08001; C12Y 402/01017

(57) ABSTRACT

A method of producing polyhydroxyalkanoate (PHA) copolymers comprising both short-chain-length (scl) and medium-chain-length (mcl) subunits, wherein some of the mcl subunits bear reactive vinyl groups, is provided. The method comprises providing cells comprising (i) a PHA synthase (phaC) gene encoding a particular class I poly(3-hydroxyalkanoate) polymerase, and (ii) a phaJ gene encoding a particular (R)-specific enoyl-CoA hydratase, and cultivating said cells in a growth medium comprising an alkenoic acid. In one example, cells grown in the presence of a mixture of decanoic acid and undecen-10-enoic acid yielded a copolymer comprising units of 3-hydroxybutyrate (94.73 mol %), 3-hydroxyhexanoate (1.51 mol %), 3-hydroxyoctanoate (2.33 mol %), 3-hydroxydecanoate (0.69 mol %) and 3-hydroxyhept-6-enoate (0.73 mol %). The presence of acrylic acid as a 13-oxidation inhibitor in the growth medium led to an increase in both the mcl-PHA subunit content and vinyl subunit content. For example, a copolymer containing about 1.2 mol % 3-hydroxyhept-6-enoate and about 0.5 mol % 3-hydroxynon-8-enoate was obtained at a concentration of 40 mM acrylic acid. PHA accumulation of up to 57.2% (w/w of cell dry weight) are reported. The scl-mcl PHAs with subunits bearing reactive vinyl groups are expected to be useful in a variety of applications, for instance for covalently linking bioactive molecules.

### CROSS-REFERENCE TO RELATED APPLICATION

This application claims benefit of European Provisional Patent Application No. EP20201070.8, which was filed in the European Patent Office on Oct. 9, 2020, the entire contents of which are incorporated herein by reference for all purposes.

### FIELD OF THE INVENTION

The present invention relates to biopolymers and their production. In particular, the invention relates to short-chain-length and short-chain-length-medium-chain-length poly-3-hydroxyalkanoates and their microbial production.

### BACKGROUND OF THE INVENTION

Poly-3-hydroxyalkanoates (PHAs) are a family of polyesters accumulated by a wide variety of microorganisms possibly as a method of conserving carbon and energy or controlling intracellular redox conditions. PHAs are subclassified based on the length of the constituent monomers. Polymers of 3-hydroxypropanoic acid, 3-hydroxybutanoic acid, and 3-hydroxypentanoic acid (C3-C5) are termed short-chain-length (scl) PHAs.

Polymers of 3-hydroxy-alkanoic acids having 6 to 14 carbon atoms, i.e. 3-hydroxyhexanoic acid to 3-hydroxytetradecanoic acid, are termed medium-chain-length (mcl) PHAs. The length of the sidechains imparts varying mechanical, physical, and chemical properties of the PHAs.

PHAs have been shown to have biomedical applications as they degrade slowly in animal tissues releasing relatively little acidity or toxic metabolites. Many of these applications require or are enhanced by slow release of bioactive compounds that have been doped into the PHA (Zhang et al. 2018). A scl-mcl PHA copolymer has been used to form PHA nanoparticles for the treatment of systemic lupus erythematosus in mice with reduced side effects (Hu et al. 2020). Another area of potential application is in wound healing, where slow release of therapeutics is required. Giourieva et al. 2019 reports on such a study where doping of the PHA was employed.

B. cepacia IPT64 has been shown to accumulate scl-PHA with unsaturated scl subunits (3-hydroxy-4-pentenoate) from unrelated carbon sources but produced more scl-PHA when fed 4-pentenoic acid (de Andrade Rodrigues et al. 2000). Rhodospirillum rubrum has also been shown to accumulate 3-hydroxy-4-pentenoate subunits in a scl-PHA copolymer with 3-hydroxybutyric acid and 3-hydroxypentanoic acid when fed 4-pentenoic acid (Ulmer et al. 1994, Ballistreri et al. 1995, Bear et al. 1997). However, 4-pentenoic acid is expensive, making it unsuitable for many or most commercial applications. In addition, it leads to the production of only short side-chains which may not be suitable for further chemical modification. A recombinant bacterial strain that can accumulate a copolymer of 3-hydroxybutanoic acid with 9-12% 3-hydroxy-dec-9-enoate has also been disclosed (CN 109266597 A).

Many of the potential applications outlined above could be based on scl-mcl PHAs. Accordingly, the availability of such scl-mcl PHAs with subunits bearing reactive groups would be very useful because this allows for covalently linking bioactive molecules to the polymer. However, to date, there has been no disclosure of a practical method of producing scl or scl-mcl PHAs with such reactive groups. Furthermore, the disclosure of CN 109266597 A provides only for a means of incorporating a C10 unsaturated subunit in the PHA using 9-decenol as substrate. These PHAs may not be suitable for all such applications, and the methods would possibly be quite expensive to carry out due to the high cost of substrates such as 9-decenol. Furthermore, the strain of CN 109266597 A is capable of accumulating only a small percentage of the cell dry weight (CDW) in PHA (e.g. 11.3% PHA of CDW), which may be a great disadvantage for large scale production. The strain of CN 109266597 A is not native with respect to the phaC gene, which encodes PHA synthase.

Cupriavidus necator, formerly known by many different names including Alcaligenes eutrophus, is the best-known producer of poly-3-hydroxyalkanoates (PHAs). It was believed to only be able to synthesize homopolymers of 3-hydroxybutyric acid until methods were found to produce poly-3-hydroxybutyric-hydroxyvaleric (P(HB-HV)) copolymers (Ramsay et al. 1990). Cupriavidus necator was chosen for the original ICI P(HB-HV) production process because of its high yield and rapid production rate. (P. A. Holmes, S. H. Collins, and W. F. Wright, European patent 69,497, April 1987).

The final step in PHA synthesis is the polymerization step, catalyzed by the enzyme, PHA synthase (PhaC). The crude C. necator synthase was believed to accept substrates no larger than C5 (Haywood et al., 1989) until several years later when it was determined that the necator synthase could incorporate some 3-hydroxyhexanoate (3HHx) (Dennis et al. 1998). Then, by cloning a phaC into β-oxidation impaired E. coli, it was found that the Cupriavidus necator synthase could accumulate small amounts of mcl-PHAs including HO (C8) and HDD (C12), but surprisingly no HD (C10) when fed octanoate, decanoate and dodecanoate. This is the only report of such activity.

To this day, it is generally believed that the Cupriavidus necator PHA synthase specificity is restricted to smaller molecular weight substrates. For example, Mezzolla et al. 2018 states that “The grouping of phaC enzymes into four classes is dependent on substrate specificity, according to the preference in forming short-chain-length (scl) or medium-chain-length (mcl) polymers: Class I, Class III and Class IV produce scl-PHAs depending on propionate, butyrate, valerate and hexanoate precursors”. The Cupriavidus necator PHA synthase is a Class I synthase.

It is also believed that PHAs comprising terminal alkene moieties on the side chains are only accumulated by mcl-PHA producers, such as P. putida. For example, Mezzolla et al. 2018 states that “There also exist PHAs with unsaturated monomers (3-hydroxyalkenoates), produced by Pseudomonas sp. possessing Class II PHA polymerizing enzymes”. While there has been one report of a Class I synthase polymerizing small amounts of 3-hydroxy-4-pentenoate (i.e. a scl-PHA) (de Andrade Rodrigues et al. 2000), production of scl-mcl-PHA copolymers (wherein the mcl subunits comprise vinyl groups) using the Cupriavidus necator PHA synthase has never been reported.

Accordingly, there is a need for better methods to produce novel scl-mcl PHAs having scl saturated subunits and mcl subunits bearing reactive vinyl groups. Furthermore, if PHAs are to replace non-sustainable or environmentally damaging plastics, there is a need for more efficient methods of production.

### SUMMARY OF THE INVENTION

The present disclosure provides for novel PHAs and methods of producing such. The inventors have surprisingly found that the native PHA synthase of C. necator is capable of polymerising both scl and mcl subunits to form a copolymer. The present inventors have further found that successful biosynthesis of the disclosed PHAs rely on incorporation of a phaJ gene encoding a broad-specificity (R)-specific enoyl-CoA hydratase.

One aspect of the disclosure provides for a polyester copolymer comprising:
a. saturated short-chain-length hydroxyalkanoate subunits, and
b. 3-hydroxyhept-6-enoate and/or 3-hydroxynon-8-enoate subunits.

One aspect of the disclosure provides for a polyester copolymer comprising:
a. 85.0-99.0 mol % of one or more saturated short-chain-length hydroxyalkanoate subunit, and
b. 0.5-5 mol % of one or more medium-chain-length 3-hydroxyalkenoate subunits,
   wherein the medium-chain-length 3-hydroxyalkenoate subunits contains a terminal0 alkene moieties in the side chain.

One aspect of the present disclosure provides for a cell capable of producing the polyester copolymers of the present disclosure, said cell comprising:
a. a phaC gene encoding a class I poly(3-hydroxyalkanoate) polymerase having at least 90% sequence identity to SEQ ID NO: 1, and
b. a phaJ gene encoding an (R)-specific enoyl-CoA hydratase having at least 90% sequence identity to SEQ ID NO: 2.

A final aspect of the disclosure provides for a method of producing a polyester copolymer as disclosed herein, said method comprising the steps of:
a. providing a cell according to the disclosure, and
b. cultivating said cell in a growth medium comprising an alkenoic acid, thereby obtaining the polyester copolymer.

The cells as disclosed herein provide a high yield of polyester product as expressed by the percentage polyester copolymer of cell-dry-weight (CDW) of the polyester copolymer.

The vinyl moieties of the mcl subunits can be advantageous for further functionalization of the polyester copolymers of the disclosure. As the copolymers of the present disclosure comprise 0.1-20.0 mol % subunits having terminal vinyl groups on the side chains, the polyester copolymers of the present disclosure are particularly useful for production of functionalized PHAs wherein a functionalization degree of at least 0.1 is required.

### DETAILED DESCRIPTION OF THE DRAWINGS

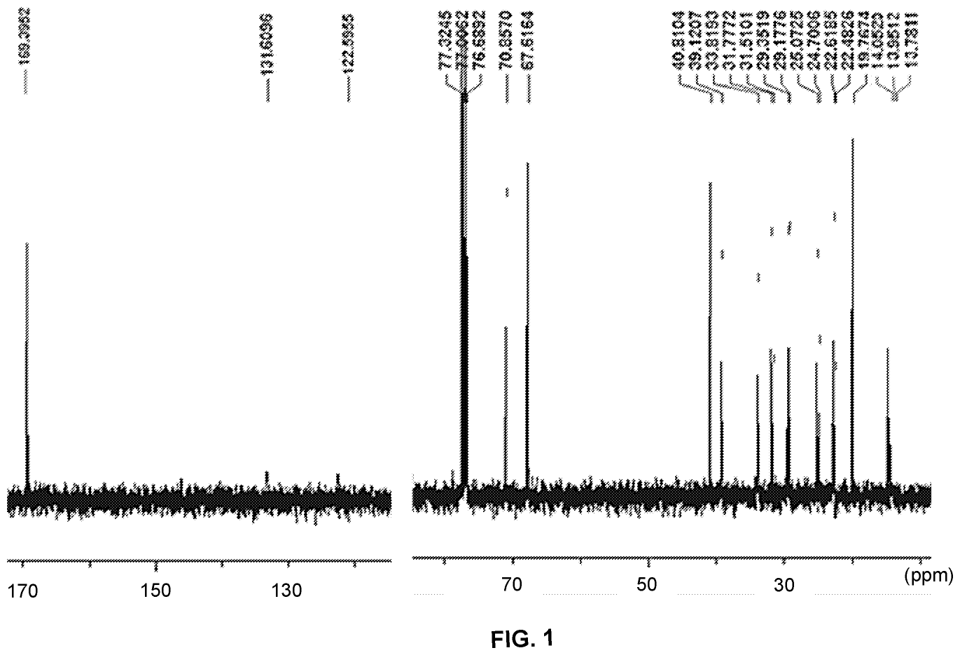

FIG. 1 shows the NMR analysis of PHA extracted from C. necator H16/pMPJAS03 biomass at the end of the fed-batch fermentation showing the composition to be 3HB (94.7 mol %)-co-3HHx (1.5 mol %)-co-3HHO (2.33 mol %)-co-3HD (0.69 mol %)-co-3HHp(=) (0.73 mol %).

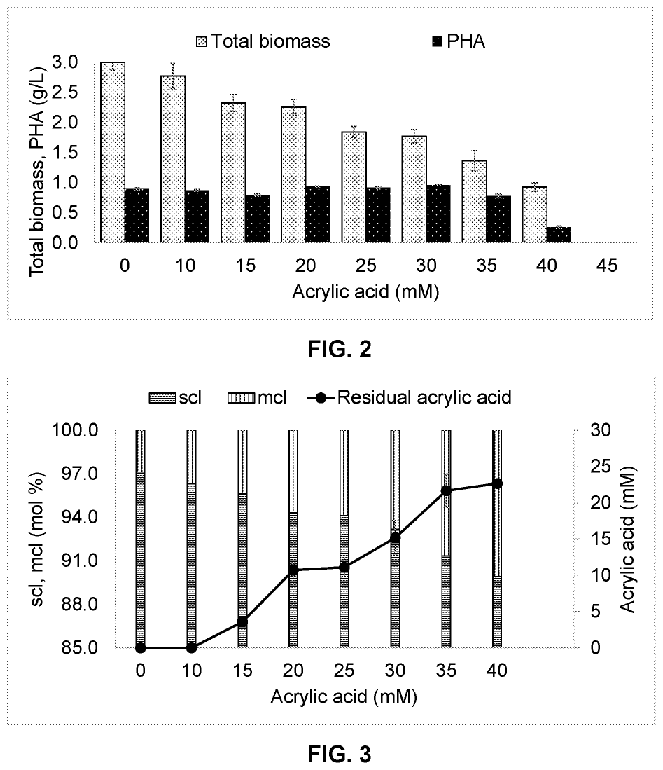

FIG. 2 shows the effect of acrylic acid addition on C. necator H16/pMPJAS03 biomass and PHA production in a phosphate-limited chemostat culture (dilution rate=0.14 h⁻¹).

FIG. 3 illustrates the effect of acrylic acid addition on the scl/mcl PHA content of C. necator H16/pMPJAS03 in a phosphate-limited chemostat culture (dilution rate=0.14 h⁻¹).

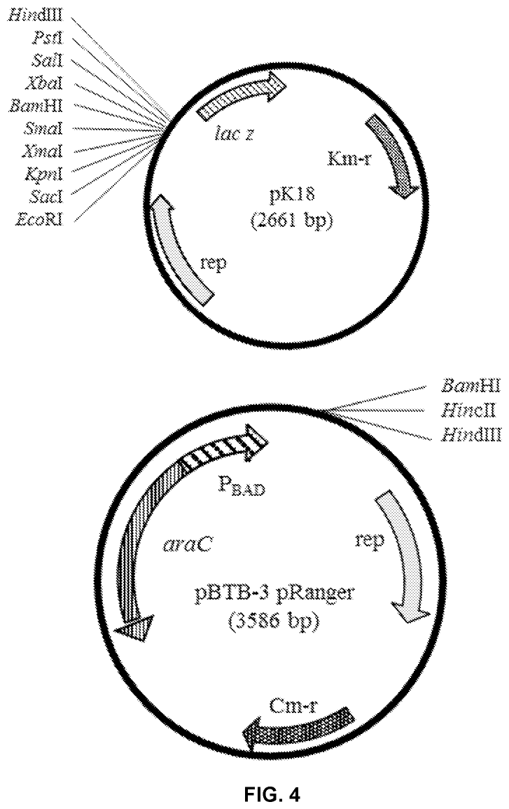

FIG. 4 illustrates a pK18 plasmid and pBTB-3 pRanger.

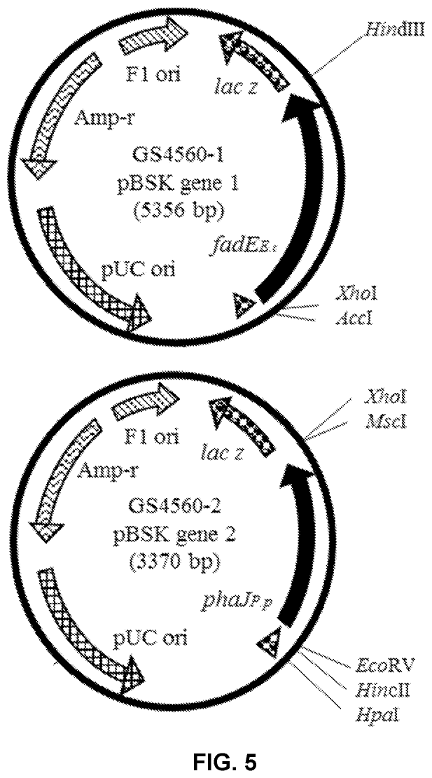

FIG. 5 illustrates a GS4560-1 pBSK gene 1 (fadE E. coli) plasmid and GS4560-2 pBSK gene 2 (phaJ P. putida) plasmid.

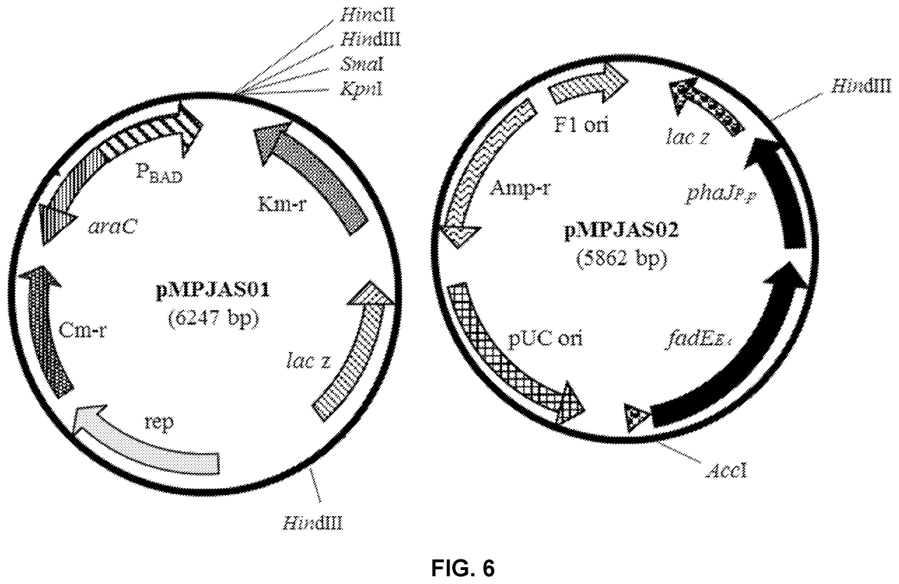

FIG. 6 illustrates a pMPJAS01 plasmid and pMPJAS02 plasmid.

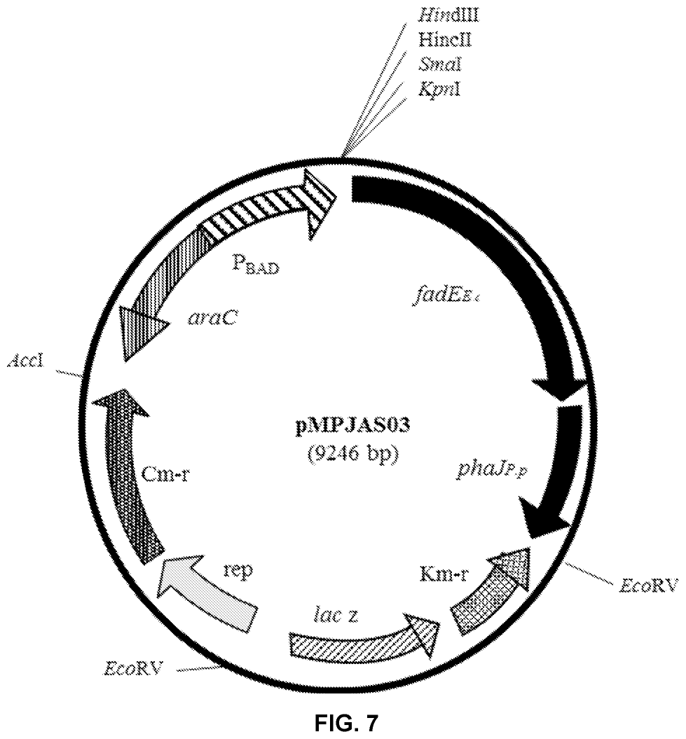

FIG. 7 illustrates a pMPJAS03 plasmid.

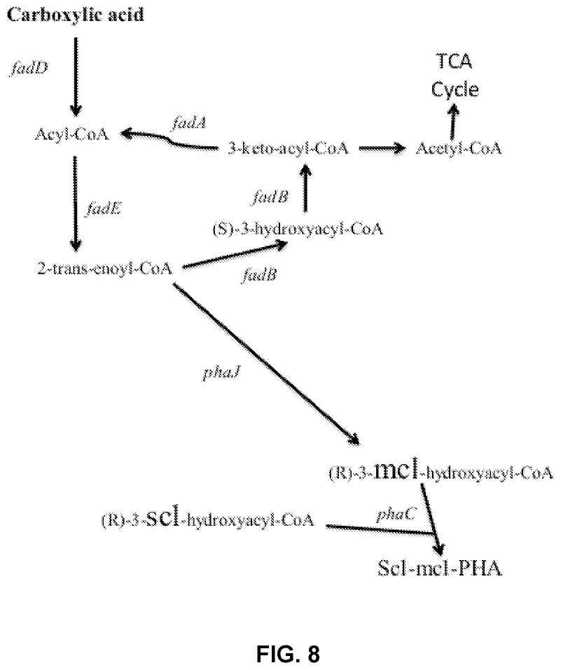

FIG. 8: depicts flowchart of the process to prepare scl/mcl PHA. (R)-3-scl-hydroxyacyl-CoA is accepted by the C. necator phaC gene product (PHA synthase) but the wild type phaJ product cannot supply sufficient (R)-3-mcl-hydroxyacyl-CoA. By replacing the wild type phaJ with one able to supply (R)-3-mcl-hydroxyacyl-CoA, allows production of scl-mcl PHA. If the mcl-hydroxyacyl-CoA supplied has a vinyl group, the vinyl group will be incorporated into the growing polymer at the end of a side-chain.

### DEFINITIONS

The abbreviation “PHA” as used herein is taken to mean polyhydroxyalkanoate, in particular poly-3-hydroxyalkanoate. They may be either homopolymers or copolymers.

The following abbreviations are used herein for PHA monomeric units: 3HB (3-hydroxybutyric acid), 3HHx (3-hydroxyheaxanoic acid), 3HHO (3-hydroxyoctanoic acid), 3HD (3-hydroxydecanoic acid) and 3HHp(=) (3-hydroxyheptenoic acid).

As used herein, the term (=) is used in relation to monomeric subunits to signify that the subunit in question comprises a terminal alkene on its side-chain.

Cupriavidus necator has previously also been called Ralstonia eutropha as well as other names. The present invention relates to the bacterial species now termed Cupriavidus necator.

As used herein, “subunit” refers to the discrete repeating molecular moiety constituting a polymer backbone. Herein, the subunits may be identified by referring to the non-polymerised monomeric unit from which the polymer was produced. By way of example, the subunits of a PHA may be referred to as 3-hydroxyalkanoate subunits or 3-hydroxyalkanoic acid subunits.

The term “mol %” in connection with the content of a subunit in a PHA refers to the amount of said subunit compared to the total amount of subunits in the PHA. By way of example, a PHA wherein 1 in every 10 subunits is of the type A would be considered to comprise 10 mol % of A.

As used herein, the term “short chain length” hydroxyalkanoate subunit refers to hydroxy-alkanoate subunits having between 3 and 5 carbon atoms. Specific examples are 3-hydroxypropanoate subunits, 3-hydroxybutanoate subunits, and 3-hydroxypentanoic subunits. That is, scl-subunits in a PHA polymer will have no sidechain, or a sidechain being 1 or 2 carbon atoms long. scl 3-hydroxyalkanoate subunits have the structure of formula (I):

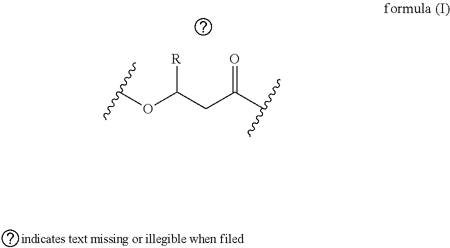

where R is H, methyl, or ethyl, and the dotted lines indicate attachment point to other subunits of the PHA.

The term “scl-hydroxyalkenoate” subunit refers to a subunit having 5 carbon atoms and one carbon-carbon double bond between carbon atoms of the side chain. An example of a scl-hydroxyalkenoate subunit is a subunit based on 3-hydroxypentenoate, i.e. a subunit having formula (II):

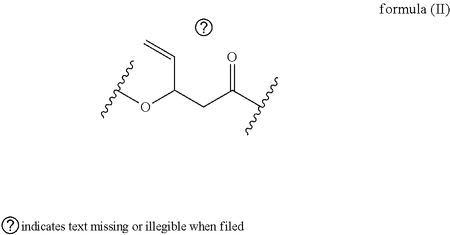 [SMILES: *OC(C=C)CC(*)=O]

where the dotted lines indicate attachment point to other subunits of the PHA.

As used herein, the term “medium chain length” hydroxyalkanoate subunit refer to hydroxy-alkanoate subunits having between 6 and 14 carbon atoms. Preferably, as used herein, the term refers to 3-hydroxyalkanoate subunits having the structure of formula (III):

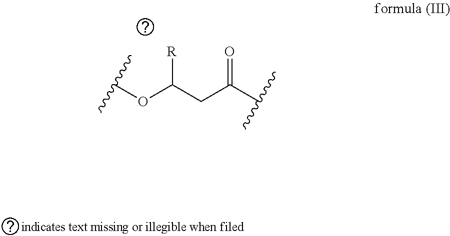

where R is an alkyl group having 2 to 10 carbon atoms, and the dotted lines indicate attachment point to other subunits of the PHA. Preferably, the alkyl group is a linear alkyl group.

The term “mcl-hydroxyalkenoate” subunit refers to a hydroxyalkenoate subunit having between 6 and 15 carbon atoms and at least one terminal carbon-carbon double bond between carbon atoms of the side chain. An example of a mcl-hydroxyalkenoate subunit is a subunit based on 3-hydroxyhept-6-enoate. i.e. a subunit having formula (IV):

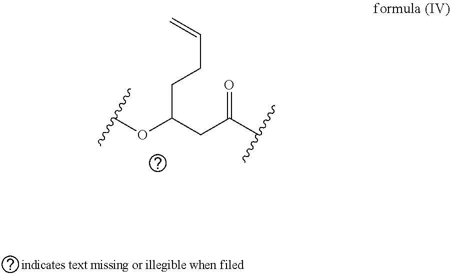 [SMILES: *OC(CCC=C)CC(*)=O]

where the dotted lines indicate attachment point to other subunits of the PHA. The side chain of the subunit may be branched or linear, but it is preferably linear. While the side chain may comprise multiple terminal double bonds, the side chain preferably comprise exactly one terminal double bond.

Phrases such as “X comprises in the range of n to m of Y” as used herein refer to that X contains at least n and at the most m of Y. I.e. the term indicates that X does not contain more than m of Y. By way of example, if a polyester material is stated to comprise in the range of 0.5 to 5.0% mcl PHA, then said polyester material does not contain more than 5.0% mcl PHA.

PHA synthase and poly(3-hydroxyalkanoate) polymerase are using interchangeably herein.

The phrase “X % of Y in cell dry weight” is taken to mean that dry biomass obtained from the cells of the disclosure comprise by weight X % of Y.

The terms “type” and “class” are synonymous for the discussion of PHA synthases, for example, a type I PHA synthase is synonymous to a class I PHA synthase.

Poly-3-hydroxyalkanoates

The present disclosure regards polyester copolymers, in particular, poly-3-hydroxyalkanoates. PHA polymers are thermoplastics and can be processed by conventional processing equipment. Depending on the specific composition, they may be ductile and elastic or hard and brittle, allowing for a wide range of applications.

One embodiment of the present disclosure provides for a polyester copolymer comprising:
a. saturated short-chain-length hydroxyalkanoate subunits, and
b. 3-hydroxyhept-6-enoate and/or 3-hydroxynon-8-enoate subunits.

The physical-mechanical properties of a PHA depend on the specific PHA subunits, and thus on the specific side chains of said subunits. scl-PHAs tend to have high crystallinity, high melting temperatures, moderate glass transition temperatures compared to the mcl-PHA counterparts. scl-PHAs also tend to have comparatively high tensile strength and high Young's modulus compared to mcl-PHAs. Finally, mcl-PHAs tend to have higher elongation at break compared to scl-PHAs. For many applications, it may be favourable for the PHA to possess the physical-mechanical properties of a scl-PHA, while also allowing for functionalisation. This can be achieved by having predominantly scl-PHA subunits present in the copolymer. Thus, one embodiment of the present disclosure provides for a polyester copolymer comprising:
a. 85.0-99.0 mol % of one or more saturated short-chain-length hydroxyalkanoate subunit, and
b. 0.5-5 mol % of one or more medium-chain-length 3-hydroxyalkanoate subunits, wherein the medium-chain-length 3-hydroxyalkanoate subunits contains an alkene moiety in the side chain.

In a preferred embodiment of the disclosure, the alkene moiety is a terminal alkene moiety.

PHAs are typically based on either 3-hydroxyalkanoic acid or 4-hydroxyalkanoic acid subunits. The PHA synthase of the present disclosure preferably polymerises 3-hydroxyalkanoate subunits. Accordingly, in one embodiment of the disclosure, the polyester copolymer comprises saturated short-chain-length polyhydroxyalkanoate subunits which are saturated short-chain-length 3-hydroxyalkanoate subunits.

PHAs based on 3-hydroxybutyric acid tend to be compostable, and thus are attractive compared to other commercially available thermoplastics. 3-hydroxybutyric acid is a scl-PHA subunit. Accordingly, in one embodiment of the present disclosure, the saturated short-chain-length 3-hydroxyalkanoate subunits is 3-hydroxybutyrate subunits.

In the context of the present disclosure, it is considered to be favourable that the side chains comprising the terminal alkene moiety are of some length. This distances the terminal alkene moiety somewhat from the PHA backbone, which in turn can make functionalisation more facile because the reactive moiety, namely the alkene group, is removed from the backbone of the PHA. mcl-PHA subunits having side chains approximately 4-6 carbon atoms in length are considered to be especially favourable for achieving this. Accordingly, in one embodiment of the present disclosure, the polyester copolymer comprises 3-hydroxyhept-6-enoate subunits. In a specific embodiment of the present disclosure, the polyester copolymer comprises:
a. saturated short-chain-length hydroxyalkanoate subunits, and
b. 3-hydroxyhept-6-enoate subunits.

In a one embodiment of the disclosure, the polyester copolymer comprises 3-hydroxynon-8-enoate subunits. In a specific embodiment of the present disclosure, the polyester copolymer comprises:
a. saturated short-chain-length hydroxyalkanoate subunits, and
b. 3-hydroxynon-8-enoate subunits.

In a further embodiment of the disclosure, the polyester copolymer comprises:
a. 60.0-99.5 mol % saturated short-chain-length hydroxyalkanoate subunits, and
b. 0.5-40.0 mol % 3-hydroxyhept-6-enoate and/or 3-hydroxynon-8-enoate subunits.

In yet a further embodiment of the disclosure, the polyester copolymer comprises:
a. 70.0-99.5 mol % saturated short-chain-length hydroxyalkanoate subunits, and
b. 0.5-30.0 mol % 3-hydroxyhept-6-enoate and/or 3-hydroxynon-8-enoate subunits.

In an even further embodiment of the present disclosure, the polyester copolymer comprises:
a. 80.0-99.5 mol % saturated short-chain-length hydroxyalkanoate subunits, and
b. 0.5-20.0 mol % 3-hydroxyhept-6-enoate and/or 3-hydroxynon-8-enoate subunits.

In yet an even further embodiment of the present disclosure, the polyester copolymer comprises:
a. 90.0-99.5 mol % saturated short-chain-length hydroxyalkanoate subunits, and
b. 0.5-10.0 mol % 3-hydroxyhept-6-enoate and/or 3-hydroxynon-8-enoate subunits.

The PHAs of the present disclosure comprise alkene moieties which are useful for functionalising said PHA with other compounds. These compounds can be bioactive compounds, which may in turn be large or bulky. The bioactive compounds might exhibit reduced activity, if they are forced close to other moieties, or for instance if their substrate cannot properly access the bioactive compound. Accordingly, it is preferred that the PHA does not comprise too many sites which are suitable for ligation of bioactive compounds, i.e. terminal alkenes, because functionalisation of the PHA might then produce a bioactive material with suboptimal activity. If release of the bioactive compounds is achieved by cleavage with an enzyme as outlined below, having a high degree of alkene-moieties or functionalised alkene moieties may also reduce the activity of the enzyme. Thus, in a preferred embodiment of the present disclosure, the polyester copolymer comprises:
a. 95.0-99.5 mol % saturated short-chain-length hydroxyalkanoate subunits, and
b. 0.5-5.0 mol % 3-hydroxyhept-6-enoate and/or 3-hydroxynon-8-enoate subunits.

### Functionalisation of PHAs

Many bioactive compositions comprising polymeric materials—so-called “bioactive polymers”—are rendered bioactive by doping (mixing) of the bioactive component with the polymeric material. This can lead to difficulties in controlling the rate of release of the bioactive component from the polymeric material. Instead, covalently linking the bioactive component to the polymeric material may provide for more stable compositions, and the rate of release of the bioactive component may be better controlled by, for example, cleavage of a cleavable linker, or by enzymatic degradation of the polymeric material. The vinyl moieties of the presently disclosed PHAs may feasibly be used for covalent attachment of bioactive components such as drugs, enzymes, and hormones. Release of the bioactive component can be affected by depolymerizing enzymes which cleave the PHA, i.e. an esterase such as a PHA depolymerase or other esterases that degrade PHAs and are found in plant or animal tissues. This can provide for better control of release rate of the bioactive component, as release is dependent on cleavage of covalent bonds rather than diffusion. Alternatively, it is desired that the bioactive component is not released from the polymeric material, i.e. the polymeric material acts a support for the bioactive component. In this case, it is advantageous to covalently link the bioactive component to the polymer to obtain an irreversibly linked bioactive component. Such a material may prove more stable than polymeric materials doped with a bioactive component, because the bioactive component may leak from the polymer. The vinyl moieties of the presently disclosed PHAs may feasibly be used for covalent attachment of bioactive components.

### Cells for Production of PHAs

The PHAs of the present disclosure may be produced using a suitable cell culture. The present inventors have shown that biosynthesis of the scl-mcl-PHA copolymers of the present disclosure can be achieved by a cell comprising a phaC gene encoding a poly(3-hydroxyalkanoate) polymerase and a phaJ gene encoding an (R)-specific enoyl-CoA hydratase. Thus, one embodiment of the present disclosure provides for a cell comprising:
a. a phaC gene encoding a class I poly(3-hydroxyalkanoate) polymerase having at least 90% sequence identity to SEQ ID NO: 1, and
b. a phaJ gene encoding an (R)-specific enoyl-CoA hydratase having at least 90% sequence identity to SEQ ID NO: 2,

wherein the cell is capable of producing the polyester copolymer of the present disclosure. In a further embodiment of the present disclosure, the cell is Cupriavidus necator.

The present inventors have found that the specific Cupriavidus necator H16 strain is useful for production of the cells of the present disclosure. Accordingly, in one embodiment of the present disclosure, the cell has been produced from the Cupriavidus necator H16 strain.

phaC Gene and PHA Synthase

It has been generally thought that biosynthesis of scl-mcl-PHA copolymers could not be carried out in cell lines having only a phaC gene encoding a class I poly(3-hydroxyalkanoate) polymerase, especially in Cupriavidus necator. However, the present inventors have found that the phaC gene native to Cupriavidus necator encodes a class I poly(3-hydroxyalkanoate) polymerase which is capable of producing scl-mcl-PHA copolymers with vinyl side-chains. Accordingly, in one embodiment of the present disclosure, the phaC gene encoding a class I poly(3-hydroxyalkanoate) polymerase is the phaC gene native to the Cupriavidus necator. In a specific embodiment of the present disclosure, the phaC gene encodes a class I poly(3-hydroxyalkanoate) polymerase that has at least 90% sequence identity to SEQ ID NO: 1, such as at least 91%, 92%, 93%, 94%, 95%, 96%, 97%, 98%, or 99% sequence identity.

phaJ Gene and (R)-Specific Enoyl-CoA Hydratase

The present inventors have found that it is paramount that the cell comprises a phaJ gene encoding a broad specificity (R)-specific enoyl-CoA hydratase. (R)-specific enoyl-CoA hydratase catalyses the synthesis of (2E)-enoyl-CoA from (3R)-hydroxyacyl-CoA, which is a vital step in the biosynthesis of 3-hydroxyalkanoates. It is paramount that the (R)-specific enoyl-CoA hydrate accepts both short- and medium chain length substrates such that it may produce scl-3-hydroxyalkanoates, mcl-3-hydroxyalkanoates, and mcl-3-hydroxyalkenoates. Thus, in one embodiment of the present disclosure, the (R)-specific enoyl-CoA hydratase is a broad specificity (R)-specific enoyl-CoA hydratase. The phaJ gene native to Cupriavidus necator encodes an (R)-specific enoyl-CoA hydratase which is capable of polymerising only scl substrates. Accordingly, in one embodiment of the disclosure, the phaJ gene encoding a broad specificity (R)-specific enoyl-CoA hydratase is not native to the cell. The phaJ gene may correspond to the gene present in Pseudomonas putida, specifically the KT2440 strain. Thus, in one embodiment of the present disclosure, the phaJ gene encoding a broad specificity (R)-specific enoyl-CoA hydratase is the phaJ gene from the Pseudomonas putida KT2440 strain. The coding sequence for the (R)-specific enoyl-CoA hydratase of Pseudomonas putida KT2440 is provided herein as SEQ ID NO: 4. The broad specificity (R)-specific enoyl-CoA hydratase encoded by this gene is provided herein as SEQ ID NO: 2. In one embodiment of the present disclosure, the phaJ gene encodes a (R)-specific enoyl-CoA hydratase that has at least 90% sequence identity to SEQ ID NO: 2, such as at least 91%, 92%, 93%, 94%, 95%, 96%, 97%, 98%, or 99% sequence identity.

fadE Gene

The fadE gene encodes acyl-CoA dehydrogenase, which catalyses the reaction of 2,3-saturated alkanoic acids to enoyl-CoA, i.e. the substrate of (R)-specific enoyl-CoA hydratase. Accordingly, it may be favourable to incorporate a fadE gene in the cell of the present disclosure to ensure a sufficient supply of substrate for (R)-specific enoyl-CoA hydratase. For some purposes, the (R)-specific enoyl-CoA hydratase may have enough substrate without the fadE gene product present, and accordingly the fadE gene may be beneficial for the biosynthesis of the present PHAs, but it is not always essential. Accordingly, in one embodiment of the present disclosure, the cell comprises a fadE gene encoding an acyl-CoA dehydrogenase. In a specific embodiment of the present disclosure, the fadE gene encodes an acyl-CoA dehydrogenase having at least 90% sequence identity to SEQ ID NO: 3. In one embodiment of the present disclosure, the fadE gene has at least 90% sequence identity to SEQ ID NO: 5. In a preferred embodiment of the present disclosure, the fadE gene is that native to the Escherichia coli K12 strain.

### Carbon Sources

The cell of the present disclosure is capable of using alkenoic acids as carbon source. Specifically, it is considered that the cell of the disclosure is capable of using alkenoic acids having terminal alkenes. Examples of alkenoic acids are hex-5-enoic acid, hept-6-enoic acid, oct-7-enoic acid, non-8-enoic acid, dec-9-enoic acid, undec-10-enoic acid, and/or dodec-11-enoic acid. The cell of the present disclosure has been shown to use undec-10-enoic acid as a carbon source. Undec-10-enoic acid is converted to mcl-3-hydroxyalkenoates and incorporated into the PHA copolymers of the disclosure. Undec-10-enoic acid is an inexpensive reagent. Thus, in a preferred embodiment of the present disclosure, the cell is capable of using undec-10-enoic acid.

The disclosed cell is capable of using fructose as a sole carbon source. Undec-10-enoic acid is desired for the production of the PHA copolymers of the present disclosure. Thus, in one embodiment of the present disclosure, the cell is capable of using undec-10-enoic acid and fructose as a carbon source.

13-oxidation Inhibition

13-oxidation mediates truncation of the undec-10-enoic of the growth and production medium to mcl-3-hydroxyalkanoate (C7 and C9). Accordingly, it may be an advantage that some 13-oxidation occurs such that the undec-10-enoic is converted to the desired substrates. However, 13-oxidation can also affect further conversion of the desired substrates 3-hydroxyhept-6-enoate and 3-hydroxynon-8-enoate, effectively reducing the amount of mcl-3-hydroxyalkenoate substrate available to the cell. The present inventors have found that some level of 13-oxidation inhibition is increases the yield of unsaturated subunits from undecenoic acid to obtain the scl-mcl-PHA copolymers of the disclosure. Accordingly, in one embodiment of the present disclosure, the cell growth medium comprises a 13-oxidation inhibitor. In a preferred embodiment of the disclosure, the 13-oxidation inhibitor is acrylic acid.

As 13-oxidation is both required to convert alkenoic acid into a suitable form for incorporation into the PHA, but also responsible for degradation of the desired products produced from said truncation, it is vital that a specific level of 13-oxidation activity is achieved. The level of 13-oxidation activity can be controlled by addition of an appropriate concentration of a 13-oxidation inhibitor such as acrylic acid. Thus, in one embodiment of the present disclosure, the growth medium comprises 10 to 100 mM acrylic acid. In a further embodiment of the disclosure, the growth medium comprises 10 to 20 mM acrylic acid, such as 20 to 30, such as 30 to 40, such as 40 to 50, such as 50 to 60, such as 60 to 70, such as 70 to 80, such as 80 to 90, such as 90 to 100 mM acrylic acid. In a preferred embodiment of the present disclosure, the grown medium comprises 10 to 40 mM acrylic acid.

### Accumulation of PHA Copolymer

The cells of the present disclosure are capable of accumulating a high amount of PHA copolymer. In one embodiment of the present disclosure, the cell is capable of accumulating at least 15% of the PHA of the disclosure in cell dry weight. In a further embodiment of the disclosure, the cell is capable of accumulating at least 20% of the PHA in cell dry weight. In yet a further embodiment of the present disclosure, the cell is capable of accumulating at least 25% of the PHA copolymer in cell dry weight. In an even further embodiment, the cell is capable of accumulating at least 30% of the PHA copolymer in cell dry weight.

### Methods of Producing PHA Copolymers

One embodiment of the present disclosure provides for a method of producing the polyester copolymer of the disclosure, said method comprising the steps of:
a. providing the cell as disclosed herein, and
b. cultivating said cell in a growth medium comprising an alkenoic acid, thereby obtaining said polyester copolymer.

In a further embodiment of the present disclosure, the growth medium comprises fructose. In another embodiment of the present disclosure, the growth medium comprises a 13-oxidation inhibitor. In a further embodiment, the 13-oxidation inhibitor is acrylic acid.

In one embodiment of the disclosure, the cells are fermented at 26.0 to 31.0° C., such as at 27.0 to 30.0° C., such as 27.5 to 29.5° C. In another embodiment, the cells are fermented at a pH of 6.6 to 7.1, such as 6.7 to 7.0. In one embodiment, air saturation is maintained at 20% or more, such as 25% or more, such as 30% or more.

In one embodiment of the disclosure, fructose and a mixture of alkanoic acid and alkenoic acid are fed to the cells. In one embodiment, the ratio of fructose to a mixture of alkanoic acid and alkenoic acid is 90:10 to 10:90 (mol(fructose):mol(alkanoic acid+alkenoic acid). In a preferred embodiment, the ration of fructose to the mixture of alkanoic acid and alkenoic acid is approximately 70:30 (mol(fructose):mol(alkanoic acid+alkenoic acid).

In a preferred embodiment of the disclosure, alkanoic acid and alkenoic acid is fed to the cells. In a further embodiment of the present disclosure, alkanoic acid and alkenoic acid is fed to the cells in a ratio of 97:3 to 50:50 (mol(alkanoic acid):mol(alkenoic acid)). In a further embodiment, the ratio of alkanoic acid to alkenoic acid is approximately 90:10 (mol(alkanoic acid):mol(alkenoic acid)). In a specific embodiment of the present disclosure, the alkanoic acid is decanoic acid. In another embodiment, the alkenoic acid is dec-9-enoic acid.

In one embodiment of the present disclosure, the cells are fermented in the presence of a 13-oxidation inhibitor at a concentration as outlined in the section “13-oxidation inhibition”.

### EXAMPLES

### Example 1: Production and Cultivation of Recombinant Strain

The plasmid pMPJAS03 was constructed using the broad host range p-BTB3 (Lynch et al. 2006) and pK18 (Pridmore 1987) plasmids as backbones. Both plasmids were digested with HindIII and fused to form pMPJAS01. The plasm id GS45640-1 pBSK gene 1 (GenBank Accession NC_000913) harbouring fadEEc was linearized with HindIII and blunt-ended with Klenow. Gene phaJPp from plasmid GS45640-1 gene 2 (GenBank Accession NC_002947) was released with EcoRV/XhoI, blunt-ended with Klenow. Plasmid pMPJAS02 resulted from fusing the linearized plasmid GS45640-1 pBSK gene 1 and the phaJ1Pp fragment. The fadEEc-phaJ1Pp fragment harboured in pMPJAS02 was released with AccI/HindIII, blunt-ended with Klenow, and inserted into pMPJAS01 to create the plasmid pMPJAS03. The plasmid was introduced via electroporation into C. necator H16, generating the recombinant C. necator H16/pMPJAS03. The recombinant plasmid harboured a kanamycin and chloramphenicol resistance properties, and the fadEEc-phaJ1Pp DNA fragment under the arabinose promoter (araC-PBAD). A schematic representation of pMPJAS03 plasmid construction is depicted in FIG. 7. FIGS. 4-6 shows schematic representation of the remaining plasm ids used for the construction of pMPJAS03. Plasm id construction was aided by gel electrophoresis screening.

The recombinant plasmid pMPJAS03 harbours kanamycin and chloramphenicol resistance genes and was designed to co-express fadE from E. coli strain K12 and phaJ1 from P. putida strain KT2440 genes under the arabinose promoter (araC-PBAD) (Araceli et al. 2020).

Fermentations were conducted in a 5.0 L stirred tank bioreactor (Infor HT, Bottmingen, Switzerland), with a working volume of 3.0 L. Temperature was set at 30.0° C.±1.0 and pH automatically controlled at 6.85±0.05 with the addition of NH4OH 28% (w/v) solution or KOH 2M. Dissolved oxygen was monitored with an Ingold polarographic electrode (Mettler Toledo, Hamilton Company, USA) and maintained at or above 30% of air saturation. The aeration was kept at 2.01/min through a controlled addition of air with pure oxygen. Stirring was provided by two six-blade Rushton impellers at speed of 700 rpm. CO2 was measured in the outlet gas stream with an infrared CO2 monitor (Guardian Plus, Topac Inc. Hingham, MA). LabVIEW 6.1 (National Instruments, Austin, Texas) was used to record the CO2 production (CPR g/L h) and to control the feeding of fructose and canola oil/DA with peristaltic pumps based on the mass of the reservoirs. The carbon sources were fed from separate reservoirs, one for canola oil/DA mixtures and other for concentrated fructose solution.

A 300 ml inoculum was distributed in 3 shake flasks of 500 mL and incubated at 30° C. and 200 rpm. After 24 h, the inoculum was transferred aseptically to the bioreactor. The fermentation was conducted in three stages:
Stage 1: Initial cultivation was conducted in batch mode, 2.0 g/L of fructose was used as sole carbon source.
Stage 2: Exponential cell growth. Fructose and canola oil (9:1) were fed at a specific growth rate of 0.14 h⁻¹. PH was controlled by the addition of NH4OH 28% (w/v).
Stage 3: PHA synthesis through NH4 limitation. Constant feeding was set at 2.5 g/L h of total substrates (canola oil/DA). Decanoic acid was solubilized to room temperature by adding 15% (v/v) of acetic acid before canola oil/DA mixing. The fadE and phaJ1 genes in the plasmid pMPJAS03 under the control of araC-PBAD promoter were constitutively expressed at the beginning of the stage by the addition of (v/v) of arabinose. NH4 concentration was maintained at approximately 0.4 g/L in the culture by the addition of ammonium sulfate in aqueous solution as required.

### Example 2: Continuous Culture Co-Feeding Fructose and Decanoic Acid

The inoculum medium contained per litre: 9.0 g of fructose, 3.7 g NH4SO4, 5.66 g NaH2PO4·12H2O, 2.70 g KH2PO4·7H2O, 0.4 g MgSO4·7H2O and 1.0 nutrient broth. The continuous culture mineral medium contained per litre: 1.8 g NH4SO4, 5.66 g NaH2PO4·12H2O, 2.70 g KH2PO4·7H2O, 0.4 g MgSO4·7H2O and 2.0 ml of trace element solution (10 g FeSO4·7H2O, 3.0 g CaCl2·2H2O, 0.3 g H3BO3, 0.2 g CoCl2·6H2O, 2.2 g ZnSO4·7H2O, 0.5 g MnSO2·4H2O, 0.15 g Na2MoO4·2H2O, 0.2 g NiCl2·6H2O and 1.0 g CuSO4·5H2O). Arabinose 0.1%, antibiotics, and acrylic acid were supplemented to the mineral medium. The cultivations were performed by co-feeding fructose/decanoic acid (previously solubilized at room temperature with 15% (v/v) of acetic acid).

All fermentation studies were conducted in a 2.0 L stirred tank bioreactor (Bioflo 11c, New Brunswick) with a working volume of 1.5 L at 28.5° C.±1.0 and pH automatically controlled with 2M KOH solution at pH 6.85±0.05. Dissolved oxygen was monitored with an Ingold polarographic electrode (Mettler Toledo, Hamilton Company, USA) and maintained at or above 30% of air saturation by keeping aeration of 0.5 L/min and an agitation speed of 700 rpm. The CO2 was measured in the outlet gas stream was measured with an infrared CO2 monitor (Guardian Plus, Topac Inc. Hingham, MA). LabVIEW 6.1 (National Instruments, Austin, Texas) was used to record the CO2 production (CPR g/L h) and to automatically control the fructose and fatty acids feeding with peristaltic pumps based on the mass of the reservoirs.

A 150 ml inoculum was cultivated at 30° C. and 200 rpm in a 500 mL shake flask. After 24 h, the inoculum was transferred aseptically to the bioreactor.

First, the bioreactor was operated in batch mode using the mineral salt medium above described supplemented with 10 g/L of fructose and 4.2 g/L of NH4SO4. After fructose was depleted, the system was shifted to continuous culture cultivation, with a constant dilution rate of 0.14 h⁻¹. The mineral flow rate was set at 0.21 L/h and the total substrate rate was maintained at 5.7 g/L, with a molar ratio of 0.69/0.31 of fructose and decanoic acid, respectively. Decanoic acid was supplemented with 10% of undecylenic acid in all cases. The volume of the culture was maintained at 1.5 L until reaching the steady-state. Steady-state was assumed once the DO and CPR plateau and maintained for a minimum of 5 working volumes. Reported data are the average of at least three samples taken three hours apart at each steady-state.

### Example 3: Extraction and Analysis of PHA

### Materials and Methods

### Biomass and Nutrient Analysis

Culture samples of 20 mL were taken in duplicates and centrifuged at 6000×g for 15 min. The resulting biomass pellets were washed twice with distilled water and lyophilized to assess total biomass measured as cell dry weight (CDW). Supernatants were reserved at −20° C. until nutrient consumption analysis were performed. Fructose was quantified colourimetrically by the 3,5-dinitrosalicylic acid (DNS) method. Ammonium was analyzed following the phenol-hypochlorite assay. Phosphate was measured via reduction of phosphomolybdate to molybdene blue. Total residual canola oil/undecylenic acid decanoic acid/undecylenic acid concentration was determined by gas chromatography (GC) analysis with benzoic acid as the internal standard following methanolysis.

### PHA Content Analysis Via Gas Chromatography (GC)

Fatty acid methyl esters evaluation was carried out according to the following method: 50 mg of lyophilized biomass was mixed with 2 mL of chloroform and 1 mL of methanol containing 14% (v/v) sulfuric acid and 0.2% (v/v) of benzoic acid as the internal standard. The reaction was conducted in borosilicate glass tubes with hermetic screw caps. Reagents and lyophilized biomass were mixed thoroughly in the tubes where were place in a water bath at 100±2° C. for 4 h. The tubes were cooled to room temperature, and 2 mL of distilled water was added, the resulting organic phase was later gently collected for GC analysis. 1 μL of the samples were injected into a Varian CP3900 gas chromatograph equipped with a flame ionization detector (FID). The temperature of the injector and detector were 250 and 170° C., respectively. PHA content (weight %) was determined as the percentage of the ratio of total PHA concentration to biomass. The residual biomass was then estimated as the difference between cell concentrations (CDW) and the PHA content.

### PHA Extraction and Purification

Lyophilized biomass was soaked with chloroform and incubated overnight at room temperature under constant stirring. PHA was recovered from the chloroform solution after filtration and precipitation with 10 volume of iced-cold methanol by drop wise addition. PHA was re-suspended in chloroform and precipitated with methanol twice. After the second precipitation, the residual solvent was eliminated through evaporation.

### PHA Composition Via NMR

The chemical structure of the PHAs was investigated by ¹H-NMR and ¹³C-NMR analysis. About 20 mg of PHA was dissolved in 1 mL of the deuterated chloroform (CDCl3) and further NMR spectra were recorded using a Bruker Avance II 400 spectrometer (Bruker Co., Billerica, MA). The ¹H-NMR and ¹³C-NMR spectra were obtained at 400 and 100 MHz, respectively at room temperature. Chemical shifts, such as resonance signals were given in ppm comparative to the outstanding signals of CDCl3 (internal standard) ¹H NMR: 7.26 ppm and ¹³C NMR: 77.42.

### Results

A fed-batch fermentation of C. necator H16/pMPJAS03 produced 59.7 g/L of dry biomass containing 49% PHA by weight. In the 3rd stage (PHA accumulation stage), 90 wt % canola oil and 10 wt % undecylenic acid was fed, resulting in a final polymer composition of 3HB (94.73 mol %)-co-3HHx (1.51 mol %)-co-3HHO (2.33 mol %)-co-3HD (0.69 mol %)-co-3HHp(=) (0.73 mol %), as evidenced by NMR (FIG. 1). These results demonstrate that the expression of phaJ in C. necator allows the native synthase to polymerize substrates are large as C10 and to be able to incorporate vinyl groups in subunits as large as C7. The results also showed that the C11 substrate undecylenic acid is truncated by 4 carbons through 13-oxidation before being incorporated into the polymer. It is assessed that a PHA bearing vinyl groups on 0.73 mol % of its subunits is suitable for many applications requiring bioactive PHA.

### Example 4: Inhibition of 13-Oxidation in Production of PHA-Chemostat

Addition of a 13-oxidation inhibitor such as acrylic acid resulted in increased vinyl subunit content. Increasing the acrylic acid in the feed inhibited biomass production but PHA accumulation was not affected until the highest levels tested 30 mM and higher (FIG. 2). Acrylic acid addition caused the mcl-PHA subunit content to increase from about 3 mol % when none was fed to greater than 10 mol % at a 40 mM acrylic acid feed, while the vinyl subunit content increased from 0 to 1.7 moles % (Table 1). At higher acrylic acid concentrations, some 3-hydroxynonenoate was accumulated. About 17 moles % of the undecylenic acid was converted to unsaturated subunits. Increasing the ratio of undecylenic acid to other carbon sources would likely increase vinyl subunit content further. Unlike pentenoic acid, undecylenic acid (derived from castor oil) can be obtained in large quantities at a reasonable price. This work reveals a practical economical method of producing scl-mcl PHA with vinyl subunits.

TABLE 1
PHA copolymer composition as a function of acrylic acid concentration in the feed. PHA content
and composition were determined in triplicate. Mean values and standard deviation are shown.

| | | PHA | | | | | | | |
|---|---|---|---|---|---|---|---|---|---|
| Acrylic | Total | content | Monomeric composition of PHAsª (mol %) | | | | | | |
| acid | biomass | (%, w/w | | | | | | 3HHp | 3HNo |
| (mM) | (g/L) | of CDW) | 3HB | 3HV | 3HHx | 3HO | 3HD | (=) | (=) |
| 0 | 2.9 | 29.9 | 96.9 | 0.2 | 1.1 | 1.2 | 0.6 | | |
| | (±0.2) | (±1.5) | (±0.2) | (±0.0) | (±0.0) | (±0.1) | (±0.1) | nd | nd |
| 10 | 2.7 | 31.5 | 96.0 | 0.3 | 1.2 | 1.4 | 0.9 | 0.3 | |
| | (±0.2) | (±2.6) | (±1.1) | (±0.0) | (±0.2) | (±0.3) | (±0.2) | (±0.0) | nd |
| 15 | 2.3 | 34.3 | 95.5 | 0.2 | 1.2 | 1.5 | 1.1 | 0.6 | |
| | (±0.2) | (±1.9) | (±1.9) | (±0.0) | (±0.1) | (±0.2) | (±0.1) | (±0.0) | nd |
| 20 | 2.2 | 41.5 | 94.1 | 0.2 | 1.1 | 2.3 | 1.6 | 0.6 | |
| | (±0.1) | (±2.3) | (±2.1) | (±0.0) | (±0.2) | (±0.2) | (±0.2) | (±0.0) | nd |
| 25 | 1.8 | 49.9 | 93.9 | 0.2 | 1.2 | 2.4 | 1.5 | 0.8 | |
| | (±0.3) | (±1.1) | (±2.6) | (±0.0) | (±0.1) | (±0.1) | (±0.1) | (±0.1) | nd |
| 30 | 1.7 | 54.1 | 92.9 | 0.3 | 1.3 | 2.9 | 1.6 | 0.8 | 0.3 |
| | (±0.2) | (±1.9) | (±0.7) | (±0.0) | (±0.1) | (±0.1) | (±0.1) | (±0.1) | (±0.0) |
| 35 | 1.3 | 57.2 | 91.1 | 0.2 | 1.4 | 3.4 | 2.6 | 0.9 | 0.3 |
| | (±0.2) | (±2.6) | (±1.1) | (±0.0) | (±0.1) | (±0.1) | (±0.1) | (±0.1) | (±0.0) |
| 40 | 0.9 | 28.1 | 89.6 | 0.3 | 1.7 | 3.8 | 2.9 | 1.2 | 0.5 |
| | (±0.3) | (±0.9) | (±1.3) | (±0.0) | (±0.1) | (±0.1) | (±0.1) | (±0.2) | (±0.1) |

ª3HB: 3-Hydroxybutyrate; 3HV: 3-Hydroxyvalerate; 3HHx: 3-Hydroxyhexanoate; 3HO: 3-Hydroxyoctanoate; 3HD: 3-Hydroxydecanoate; 3HHp (=): 3-Hydroxyhept-6-enoate; 3HNo (=): 3-Hydroxynon-8-enoate

### Items:

The present invention is defined in part by the following items
A polyester copolymer comprising:
   a. saturated short-chain-length hydroxyalkanoate subunits, and b. 3-hydroxyhept-6-enoate and/or 3-hydroxynon-8-enoate subunits.
A polyester copolymer comprising:
   a. 85.0-99.5 mol % of one or more saturated short-chain-length hydroxyalkanoate subunits, and
   b. 0.5-5 mol % of one or more medium-chain-length 3-hydroxyalkenoate subunits,
wherein the medium-chain-length 3-hydroxyalkenoate subunits comprise a terminal alkene moiety in the side chain.

The polyester copolymer according to any one of the preceding items, wherein the alkene moiety is a terminal alkene moiety.

The polyester copolymer according to any one of the preceding items, wherein the saturated short-chain-length polyhydroxyalkanoate subunits are saturated short-chain-length 3-hydroxyalkanoate subunits.

The polyester copolymer according to any one of the preceding items, wherein the saturated short-chain-length 3-hydroxyalkanoate subunits are 3-hydroxybutyrate subunits.

The polyester copolymer according to any one of the preceding items, comprising:
a. saturated short-chain-length hydroxyalkanoate subunits, and b. 3-hydroxyhept-6-enoate subunits.

The polyester copolymer according to any one of the preceding items, comprising:
a. saturated short-chain-length hydroxyalkanoate subunits, and b. 3-hydroxynon-8-enoate subunits.

The polyester copolymer according to any one of the preceding items, comprising:
a. 60.0-99.5 mol % saturated short-chain-length hydroxyalkanoate subunits, and
b. 0.5-40.0 mol % 3-hydroxyhept-6-enoate and/or 3-hydroxynon-8-enoate subunits.

The polyester copolymer according to any one of the preceding items, comprising:
a. 70.0-99.5 mol % saturated short-chain-length hydroxyalkanoate subunits, and
b. 0.5-30.0 mol % 3-hydroxyhept-6-enoate and/or 3-hydroxynon-8-enoate subunits.

The polyester copolymer according to any one of the preceding items, comprising:
a. 80.0-99.5 mol % saturated short-chain-length hydroxyalkanoate subunits, and
b. 0.5-20.0 mol % 3-hydroxyhept-6-enoate and/or 3-hydroxynon-8-enoate subunits.

The polyester copolymer according to any one of the preceding items, comprising:
a. 90.0-99.5 mol % saturated short-chain-length hydroxyalkanoate subunits, and
b. 0.5-10.0 mol % 3-hydroxyhept-6-enoate and/or 3-hydroxynon-8-enoate subunits.

The polyester copolymer according to any one of the preceding items, comprising:
a. 95.0-99.5 mol % saturated short-chain-length hydroxyalkanoate subunits, and
b. 0.5-5.0 mol % 3-hydroxyhept-6-enoate and/or 3-hydroxynon-8-enoate subunits.

A method of producing the polyester copolymer according to any one of the preceding items, said method comprising the steps of:
a. providing a cell comprising:
   i. a phaC gene encoding a class I poly(3-hydroxyalkanoate) polymerase having at least 90% sequence identity to SEQ ID NO: 1, and
   ii. a phaJ gene encoding an (R)-specific enoyl-CoA hydratase having at least 90% sequence identity to SEQ ID NO: 2.
b. cultivating said cell in a growth medium comprising an alkenoic acid, thereby obtaining said polyester copolymer.

14. The method according to any one of the preceding items, wherein the class I poly(3-hydroxyalkanaote) polymerase has at least 91% sequence identity to SEQ ID NO: 1, such as at least 92%, 93 OA, 94 OA, 95 OA, 96 OA, 97 OA, 98 OA, such as at least 99% sequence identity to SEQ ID NO: 1.

The method according to any one of the preceding items, wherein the (R)-specific enoyl-CoA hydratase has at least 91% sequence identity to SEQ ID NO: 2, such as at least 92%, 93%, 94%, 95%, 96%, 97%, 98%, such as at least 99% sequence identity to SEQ ID NO: 2.

16. The method according to any one of the preceding items, wherein the phaC gene is native to the cell.

17. The method according to any one of the preceding items, wherein the (R)-specific enoyl-CoA hydratase is a broad specificity (R)-specific enoyl-CoA hydratase.

18. The method according to any one of the preceding items, wherein the phaJ gene encoding a broad specificity (R)-specific enoyl-CoA hydratase is not native to the cell.

19. The method according to any one of the preceding items, wherein the phaJ gene encoding a broad specificity (R)-specific enoyl-CoA hydratase is the phaJ gene from the Pseudomonas putida KT2440 strain.

The method according to any one of the preceding items, wherein the cell is Cupriavidus necator.

21. The method according to any one of the preceding items, wherein the cell is Cupriavidus necator H16 strain.

22. The method according to any one of the preceding items, wherein the cell further comprises a fadE gene encoding an acyl-CoA dehydrogenase having at least 90% sequence identity to SEQ ID NO: 3.

23. The method according to any one of the preceding items, wherein the fadE gene encoding an acyl-CoA dehydrogenase is fadE gene from the Escherichia coli K12 strain.

24. The method according to any one of the preceding items, wherein the cell is capable of using undec-10-enoic acid as a carbon source.

The method according to any one of the preceding items, wherein the cell is capable of using undec-10-enoic acid and fructose as sole carbon source.

26. The method according to any one of the preceding items, wherein the cell is capable of accumulating at least 15% of the polyester copolymer according to any one of the preceding items in cell dry weight, such as at least 20% of the polyester.

27. The method according to any one of the preceding items, wherein the cell is capable of accumulating at least 30% of the polyester copolymer according to any one of the preceding items in cell dry weight.

28. The method according to any one of the preceding items, wherein the growth medium comprises a 13-oxidation inhibitor.

29. The method according to any one of the preceding items, wherein the 13-oxidation inhibitor is acrylic acid.

The method according to any one of the preceding items, wherein the alkenoic acid is undec-10-enoic acid.

31. The method according to any one of the preceding items, wherein the phaC gene is native to the cell.

32. The method according to any one of the preceding items, wherein the (R)-specific enoyl-CoA hydratase is a broad specificity (R)-specific enoyl-CoA hydratase.

33. The method according to any one of the preceding items, wherein the phaJ gene encoding a broad specificity (R)-specific enoyl-CoA hydratase is not native to the cell.

34. The method according to any one of the preceding items, wherein the phaJ gene encoding a broad specificity (R)-specific enoyl-CoA hydratase is the phaJ gene from the Pseudomonas putida KT2440 strain.

The method according to any one of the preceding items, wherein the cell is Cupriavidus necator.

36. The method according to any one of the preceding items, wherein the cell is Cupriavidus necator H16 strain.

37. The method according to any one of the preceding items, wherein the cell further comprises a fadE gene encoding an acyl-CoA dehydrogenase having at least 90% sequence identity to SEQ ID NO: 3.

38. The method according to any one of the preceding items, wherein the acyl-CoA dehydrogenase has at least 91% sequence identity to SEQ ID NO: 3, such as at least 92%, 93%, 94%, 95%, 96%, 97%, 98%, such as at least 99% sequence identity to SEQ ID NO: 3.

39. The method according to any one of the preceding items, wherein the fadE gene encoding an acyl-CoA dehydrogenase is fadE gene from the Escherichia coli K12 strain.

40. The method according to any one of the preceding items, wherein the cell is capable of using undec-10-enoic acid as a carbon source.

41. The method according to any one of the preceding items, wherein the cell is capable of using undec-10-enoic acid and fructose as sole carbon source.

42. The method according to any one of the preceding items, wherein the cell is capable of accumulating at least 15% of the polyester copolymer according to any one of the preceding items in cell dry weight, such as at least 20% of the polyester.

43. The method according to any one of the preceding items, wherein the cell is capable of accumulating at least 30% of the polyester copolymer according to any one of the preceding items in cell dry weight.

Sequences

SEQ ID NO: 1
Poly(3-hydroxyalkanoate) polymerase (PHA synthase):
MATGKGAAASTQEGKSQPFKVTPGPFDPATWLEWSRQWQGTEGNGHAAASGIPGL
DALAGVKIAPAQLGDIQQRYMKDFSALWQAMAEGKAEATGPLHDRRFAGDAWRTNLP
YRFAAAFYLLNARALTELADAVEADAKTRQRIRFAISQWVDAMSPANFLATNPEAQRLL
IESGGESLRAGVRNMMEDLTRGKISQTDESAFEVGRNVAVTEGAVVFENEYFQLLQYK
PLTDKVHARPLLMVPPCINKYYILDLQPESSLVRHVVEQGHTVFLVSWRNPDASMAGS
TWDDYIEHAAIRAIEVARDISGQDKINVLGFCVGGTIVSTALAVLAARGEHPAASVTLLTT
LLDFADTGILDVFVDEGHVQLREATLGGGAGAPCALLRGLELANTFSFLRPNDLVWNY
WVDNYLKGNTPVPFDLLFWNGDATNLPGPWYCWYLRHTYLQNELKVPGKLTVCGVPV
DLASIDVPTYIYGSREDHIVPWTAAYASTALLANKLRFVLGASGHIAGVINPPAKNKRSH
WINDALPESPQQWLAGAIEHHGSWWPDWTAWLAGQAGAKRAAPANYGNARYRAIE
PAPGRYVKAKA
SEQ ID NO: 2
(R)-specific enoyl-CoA hydratase:
MSQVTNTPYEALEVGQKAEYKKSVEERDIQLFAAMSGDHNPVHLDAEFAAKSMFRERI
AHGMFSGALISAAVACTLPGPGTIYLGQQMSFQKPVKIGDTLTVRLEILEKLPKFKVRIA
TNVYNQNDELVVAGEAEILAPRKQQTVELVSPPNFVAS
SEQ ID NO: 3
Acyl-coenzyme A dehydrogenase:
MMILSILATVVLLGALFYHRVSLFISSLILLAWTAALGVAGLWSAWVLVPLAIILVPFNFAP
MRKSMISAPVFRGFRKVMPPMSRTEKEAIDAGTTWWEGDLFQGKPDWKKLHNYPQP
RLTAEEQAFLDGPVEEACRMANDFQITHELADLPPELWAYLKEHRFFAMIIKKEYGGLE
FSAYAQSRVLQKLSGVSGILAITVGVPNSLGPGELLQHYGTDEQKDHYLPRLARGQEIP
CFALTSPEAGSDAGAIPDTGIVCMGEWQGQQVLGMRLTWNKRYITLAPIATVLGLAFK
LSDPEKLLGGAEDLGITCALIPTTTPGVEIGRRHFPLNVPFQNGPTRGKDVFVPIDYIIGG
PKMAGQGWRMLVECLSVGRGITLPSNSTGGVKSVALATGAYAHIRRQFKISIGKMEGI
EEPLARIAGNAYVMDAAASLITYGIMLGEKPAVLSAIVKYHCTHRGQQSIIDAMDITGGK
GIMLGQSNFLARAYQGAPIAITVEGANILTRSMMIFGQGAIRCHPYVLEEMEAAKNNDV
NAFDKLLFKHIGHVGSNKVRSFWLGLTRGLTSSTPTGDATKRYYQHLNRLSANLALLS
DVSMAVLGGSLKRRERISARLGDILSQLYLASAVLKRYDDEGRNEADLPLVHWGVQDA
LYQAEQAMDDLLQNFPNRVVAGLLNVVIFPTGRHYLAPSDKLDHKVAKILQVPNATRS
RIGRGQYLTPSEHNPVGLLEEALVDVIAADPIHQRICKELGKNLPFTRLDELAHNALVKG
LIDKDEAAILVKAEESRLRSINVDDFDPEELATKPVKLPEKVRKVEAA
SEQ ID NO: 4
Coding sequence of PhaJ gene from P. putida KT2440:
ATGAGCCAGGTGACCAACACCCCGTACGAGGCCCTGGAGGTGGGCCAGAAGGCC
GAGTACAAGAAGAGCGTGGAGGAGCGCGACATCCAGCTGTTCGCCGCCATGAGC
GGCGACCACAACCCGGTGCACCTGGACGCCGAGTTCGCCGCCAAGAGCATGTTC
GCGAGCGCATCGCCCACGGCATGTTCAGCGGCGCCCTGATCAGCGCCGCCGTGG
CCTGCACCCTGCCGGGCCCGGGCACCATCTACCTGGGCCAGCAGATGAGCTTCC
AGAAGCCGGTGAAGATCGGCGACACCCTGACCGTGCGCCTGGAGATCCTGGAGA
AGCTGCCGAAGTTCAAGGTGCGCATCGCCACCAACGTGTACAACCAGAACGACGA
GCTGGTGGTGGCCGGCGAGGCCGAGATCCTGGCCCCGCGCAAGCAGCAGACCG
TGGAGCTGGTGAGCCCGCCGAACTTCGTGGCCAGCTGA
SEQ ID NO: 5
Coding sequence of FadE gene from E. coli K12:
ATGATGATCCTGAGCATCCTGGCCACCGTGGTGCTGCTGGGCGCCCTGTTCTACC
ACCGCGTGAGCCTGTTCATCAGCAGCCTGATCCTGCTGGCCTGGACCGCCGCCCT
GGGCGTGGCCGGCCTGTGGAGCGCCTGGGTGCTGGTGCCGCTGGCCATCATCCT
GGTGCCGTTCAACTTCGCCCCGATGCGCAAGAGCATGATCAGCGCCCCGGTGTTC
CGCGGCTTCCGCAAGGTGATGCCGCCGATGAGCCGCACCGAGAAGGAGGCCATC
GACGCCGGCACCACCTGGTGGGAGGGCGACCTGTTCCAGGGCAAGCCGGACTG
GAAGAAGCTGCACAACTACCCGCAGCCGCGCCTGACCGCCGAGGAGCAGGCCTT
CCTGGACGGCCCGGTGGAGGAGGCCTGCCGCATGGCCAACGACTTCCAGATCAC
CCACGAGCTGGCCGACCTGCCGCCGGAGCTGTGGGCCTACCTGAAGGAGCACCG
CTTCTTCGCCATGATCATCAAGAAGGAGTACGGCGGCCTGGAGTTCAGCGCCTAC
GCCCAGAGCCGCGTGCTGCAGAAGCTGAGCGGCGTGAGCGGCATCCTGGCCATC
ACCGTGGGCGTGCCGAACAGCCTGGGCCCGGGCGAGCTGCTGCAGCACTACGG
CACCGACGAGCAGAAGGACCACTACCTGCCGCGCCTGGCCCGCGGCCAGGAGAT
CCCGTGCTTCGCCCTGACCAGCCCGGAGGCCGGCAGCGACGCCGGCGCCATCC
CGGACACCGGCATCGTGTGCATGGGCGAGTGGCAGGGCCAGCAGGTGCTGGGC
ATGCGCCTGACCTGGAACAAGCGCTACATCACCCTGGCCCCGATCGCCACCGTGC
TGGGCCTGGCCTTCAAGCTGAGCGACCCGGAGAAGCTGCTGGGCGGCGCCGAG
GACCTGGGCATCACCTGCGCCCTGATCCCGACCACCACCCCGGGCGTGGAGATC
GGCCGCCGCCACTTCCCGCTGAACGTGCCGTTCCAGAACGGCCCGACCCGCGGC
AAGGACGTGTTCGTGCCGATCGACTACATCATCGGCGGCCCGAAGATGGCCGGC
CAGGGCTGGCGCATGCTGGTGGAGTGCCTGAGCGTGGGCCGCGGCATCACCCTG
CCGAGCAACAGCACCGGCGGCGTGAAGAGCGTGGCCCTGGCCACCGGCGCCTA
CGCCCACATCCGCCGCCAGTTCAAGATCAGCATCGGCAAGATGGAGGGCATCGA
GGAGCCGCTGGCCCGCATCGCCGGCAACGCCTACGTGATGGACGCCGCCGCCA
GCCTGATCACCTACGGCATCATGCTGGGCGAGAAGCCGGCCGTGCTGAGCGCCA
TCGTGAAGTACCACTGCACCCACCGCGGCCAGCAGAGCATCATCGACGCCATGGA
CATCACCGGCGGCAAGGGCATCATGCTGGGCCAGAGCAACTTCCTGGCCCGCGC
CTACCAGGGCGCCCCGATCGCCATCACCGTGGAGGGCGCCAACATCCTGACCCG
CAGCATGATGATCTTCGGCCAGGGCGCCATCCGCTGCCACCCGTACGTGCTGGA
GGAGATGGAGGCCGCCAAGAACAACGACGTGAACGCCTTCGACAAGCTGCTGTTC
AAGCACATCGGCCACGTGGGCAGCAACAAGGTGCGCAGCTTCTGGCTGGGCCTG
ACCCGCGGCCTGACCAGCAGCACCCCGACCGGCGACGCCACCAAGCGCTACTAC
CAGCACCTGAACCGCCTGAGCGCCAACCTGGCCCTGCTGAGCGACGTGAGCATG
GCCGTGCTGGGCGGCAGCCTGAAGCGCCGCGAGCGCATCAGCGCCCGCCTGGG
CGACATCCTGAGCCAGCTGTACCTGGCCAGCGCCGTGCTGAAGCGCTACGACGA
CGAGGGCCGCAACGAGGCCGACCTGCCGCTGGTGCACTGGGGCGTGCAGGACG
CCCTGTACCAGGCCGAGCAGGCCATGGACGACCTGCTGCAGAACTTCCCGAACC
GCGTGGTGGCCGGCCTGCTGAACGTGGTGATCTTCCCGACCGGCCGCCACTACC
TGGCCCCGAGCGACAAGCTGGACCACAAGGTGGCCAAGATCCTGCAGGTGCCGA
ACGCCACCCGCAGCCGCATCGGCCGCGGCCAGTACCTGACCCCGAGCGAGCACA
ACCCGGTGGGCCTGCTGGAGGAGGCCCTGGTGGACGTGATCGCCGCCGACCCG
ATCCACCAGCGCATCTGCAAGGAGCTGGGCAAGAACCTGCCGTTCACCCGCCTG
GACGAGCTGGCCCACAACGCCCTGGTGAAGGGCCTGATCGACAAGGACGAGGCC
GCCATCCTGGTGAAGGCCGAGGAGAGCCGCCTGCGCAGCATCAACGTGGACGAC
TTCGACCCGGAGGAGCTGGCCACCAAGCCGGTGAAGCTGCCGGAGAAGGTGCGC
AAGGTGGAGGCCGCCTAA

### REFERENCES

de Andrade Rodrigues M F, Vicente E J, Steinbüchel A. Studies on polyhydroxyalkanoate (PHA) accumulation in a PHA synthase I-negative mutant of Burkholderia cepacia generated by homogenotization. FEMS microbiology letters. 2000 Dec. 1; 193(1):179-85.
Araceli, F. S., Arthi, R., Del Rocio, L. C. M., Berenice, V. P. and Fermín, P. G., 2020. Biosynthesis of polyhydroxyalkanoates from vegetable oil under the co-expression of fadE and phaJ genes in Cupriavidus necator. International Journal of Biological Macromolecules.
Ballistreri A, Montaudo G, Impallomeni G, Lenz R W, Ulmer H W, Fuller R C. Synthesis and characterization of polyesters produced by Rhodospirillum rubrum from pentenoic acid. Macromolecules. 1995 May; 28(10):3664-71.
Bear M M, Leboucher-Durand M A, Langlois V, Lenz R W, Goodwin S, Guérin P. Bacterial poly-3-hydroxyalkenoates with epoxy groups in the side chains. Reactive and Functional Polymers. 1997 Sep. 1; 34(1):65-77.
Dennis, D., McCoy, M., Stangl, A., Valentin, H. E. and Wu, Z., 1998. Formation of poly (3-hydroxybutyrate-co-3-hydroxyhexanoate) by PHA synthase from Ralstonia eutropha. Journal of biotechnology, 64(2-3), pp. 177-186.
Giourieva V S, Papi R M, Pantazaki A A. Polyhydroxyalkanoates Applications in Antimicrobial Agents Delivery and Wound Healing. In Biotechnological Applications of Polyhydroxyalkanoates 2019 (pp. 49-76). Springer, Singapore.
Haywood, G. W., Anderson, A. J., Williams, D. R., Dawes, E. A. and Ewing, D. F., 1991. Accumulation of a poly (hydroxyalkanoate) copolymer containing primarily 3-hydroxyvalerate from simple carbohydrate substrates by Rhodococcus sp. NCIMB 40126. International Journal of Biological Macromolecules, 13(2), pp. 83-88.
Hu J, Wang M, Xiao X, Zhang B, Xie Q, Xu X, Li S, Zheng Z, Wei D, Zhang X. A novel long-acting azathioprine polyhydroxyalkanoate nanoparticle enhances treatment efficacy for systemic lupus erythematosus with reduced side effects. Nanoscale. 2020; 12(19):10799-808.
Lynch M D, Gill R T, Broad host range vectors for stable genomic library construction. Biotechnol. Bioeng. 2006, 94, 151-158.
Mezzolla, V., D'Urso, O. F. and Poltronieri, P., 2018. Role of PhaC Type I and Type II enzymes during PHA biosynthesis. Polymers, 10(8), p. 910.
Pridmore R D, New and versatile cloning vectors with kanamycin-resistance marker. Gene, 1987, 56, 309-312.
Ramsay, B. A., Lomaliza, K., Chavarie, C., Dube, B., Bataille, P. and Ramsay, J. A., 1990. Production of poly-(beta-hydroxybutyric-co-beta-hydroxyvaleric) acids. Applied and Environmental Microbiology, 56(7), pp. 2093-2098.
Ulmer H W, Gross R A, Posada M, Weisbach P, Fuller R C, Lenz R W. Bacterial production of poly (.beta.-hydroxyalkanoates) containing unsaturated repeating units by Rhodospirillum rubrum. Macromolecules. 1994 March; 27(7):1675-9.
Zhang J, Shishatskaya E I, Volova T G, da Silva L F, Chen G Q. Polyhydroxyalkanoates (PHA) for therapeutic applications. Materials Science and Engineering: C. 2018 May 1; 86:144-50.

1. A polyester copolymer comprising:
a. 85.0-99.5 mol % of at least one saturated short-chain-length hydroxyalkanoate subunit, and
b. 0.5-5 mol % of at least one medium-chain-length 3-hydroxyalkenoate subunits, wherein the medium-chain-length 3-hydroxyalkenoate subunits comprise an alkene moiety in the side chain.

2. (canceled)

3. The polyester copolymer according to claim 1, wherein the alkene moiety is a terminal alkene moiety

4. The polyester copolymer according to claim 1, wherein the saturated short-chain-length polyhydroxyalkanoate subunits are saturated short-chain-length 3-hydroxyalkanoate subunits.

5. The polyester copolymer according to claim 4, wherein the saturated short-chain-length 3-hydroxyalkanoate subunits are 3-hydroxybutyrate subunits.

6. The polyester copolymer according to claim 1, wherein the medium-chain-length 3-hydroxyalkenoate subunits are 3-hydroxyhept-6-enoate subunits and/or 3-hydroxynon-8-enoate subunits.

7. (canceled)

8. The polyester copolymer according to claim 6, comprising:
a. 60.0-99.5 mol % of at least one saturated short-chain-length hydroxyalkanoate subunits, and
b. 0.5-40.0 mol % of 3-hydroxyhept-6-enoate subunits and/or 3-hydroxynon-8-enoate subunits.

9.-12. (canceled)

13. A method of producing the polyester copolymer of claim 1, the method comprising the steps of:
a. providing a cell comprising:
i. a phaC gene encoding a class I poly(3-hydroxyalkanoate) polymerase having at least 90% sequence identity to SEQ ID NO: 1, and
ii. a phaJ gene encoding an (R)-specific enoyl-CoA hydratase having at least % sequence identity to SEQ ID NO: 2.
b. cultivating the cell in a growth medium comprising an alkenoic acid, thereby obtaining the polyester copolymer.

14. (canceled)

15. (canceled)

16. The method according to claim 13, wherein the phaC gene is native to the cell.

17. The method according to claim 13, wherein the (R)-specific enoyl-CoA hydratase is a broad specificity (R)-specific enoyl-CoA hydratase.

18. The method according to claim 17, wherein the phaJ gene encoding a broad specificity (R)-specific enoyl-CoA hydratase is not native to the cell.

19. The method according to claim 18, wherein the phaJ gene encoding a broad specificity (R)-specific enoyl-CoA hydratase is the phaJ gene from the Pseudomonas putida KT2440 strain.

20. The method according to claim 13, wherein the cell is Cupriavidus necator.

21. The method according to claim 13, wherein the cell is Cupriavidus necator H16 strain.

22. The method according to claim 13, wherein the cell further comprises a fadE gene encoding an acyl-CoA dehydrogenase having at least 90% sequence identity to SEQ ID NO: 3.

23. The method according to claim 22, wherein the fadE gene encoding an acyl-CoA dehydrogenase is fadE gene from the Escherichia coli K12 strain.

24. The method according to claim 13, wherein the cell is capable of using undec-10-enoic acid as a carbon source.

25. (canceled)

26. The method according to claim 13, wherein the cell is capable of accumulating at least 15% of the polyester copolymer in cell dry weight.

27. (canceled)

28. The method according to claim 13, wherein the growth medium comprises a 13-oxidation inhibitor.

29. The method according to claim 28, wherein the 13-oxidation inhibitor is acrylic acid.

30. The method according to claim 13, wherein the alkenoic acid is undec-10-enoic acid.

31.-43. (canceled)
